(19) United States
(12) Patent Application Publication
(10) Pub. No.: US 2026/0260656 A1
(43) Pub. Date: Sep. 3, 2026

(54) SPEECH TO TEXT TRANSCRIPTION DEVICE

(71) Applicant: Lorena Kirby, Carlisle, KY (US)

(72) Inventor: Lorena Kirby, Carlisle, KY (US)

(21) Appl. No.: 19/068,244

(22) Filed: Mar. 3, 2025

(51) Int. Cl. G10L 15/26; G16H 10/60; H04R 1/02; H04R 1/08

(52) U.S. Cl. CPC G10L 15/26; G16H 10/60; H04R 1/02; H04R 1/08

(57) ABSTRACT

A speech to text transcription device includes a personal electronic device which has an electronic memory that stores a database comprising a speech to text translator. A housing is provided which can be worn on clothing of the medical professional without catching on the clothing. A microphone is integrated into the housing to detect the audible sound of the words spoken by the medical professional. A broadcast transceiver is integrated into the housing which is in wireless communication with the personal electronic device. The broadcast transceiver broadcasts the signal from the microphone to the personal electronic device such that the speech to text translator can translate the words spoken by the medical professional into a text based medical record which is subsequently stored in the electronic memory in the personal electronic device.

### (B) CROSS-REFERENCE TO RELATED APPLICATIONS

Not Applicable

### (C) STATEMENT REGARDING FEDERALLY SPONSORED RESEARCH OR DEVELOPMENT

Not Applicable

### (D) THE NAMES OF THE PARTIES TO A JOINT RESEARCH AGREEMENT

Not Applicable

### (E) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (F) STATEMENT REGARDING PRIOR DISCLOSURES BY THE INVENTOR OR JOINT INVENTOR

Not Applicable

### (G) BACKGROUND OF THE INVENTION

### (1) Field of the Invention

The disclosure relates to transcription devices and more particularly pertains to a new transcription device for transcribing spoken words of a medical professional into text which is stored in a personal electronic device. The device includes a personal electronic device that stores a speech to text translator and a housing that is wearable on clothing of the medical professional. The device includes a microphone that is integrated into the housing to detect words spoken by the medical professional. The device includes a broadcast transceiver that is integrated into the housing which is in wireless communication with the personal electronic device to broadcast the words spoken by the medical professional to the speech to text translator such that the speech to text translator transcribes the spoken words of the medical professional into a text based medical record.

### (2) Description of Related Art Including Information Disclosed Under 37 CFR 1.97 and 1.98

The prior art relates to transcription devices including: a wireless communication system that includes a central computer and wireless access points and personal badges that include a communication device to initiate phone calls; a variety of electronic communication devices that each at least includes a communication device that is worn on a user and a remote data storage device and an extrinsic communication network that facilitates wireless communication between the communication device and the remote data storage device. In no instance does the prior art disclose a speech to text transcription device that includes a personal electronic device that stores a speech to text translator and a housing that is wearable on a medical professional and a microphone integrated into the housing to record words spoken by the medical professional and a broadcast transceiver integrated into the housing which broadcasts the words spoken by the medical professional to the personal electronic device such that the speech to text translator converts the spoken words into a text based medical record.

### (H) BRIEF SUMMARY OF THE INVENTION

An embodiment of the disclosure meets the needs presented above by generally comprising a personal electronic device which has an electronic memory that stores a database comprising a speech to text translator. A housing is provided which can be worn on clothing of the medical professional without catching on the clothing. A microphone is integrated into the housing to detect the audible sound of the words spoken by the medical professional. A broadcast transceiver is integrated into the housing which is in wireless communication with the personal electronic device. The broadcast transceiver broadcasts the signal from the microphone to the personal electronic device such that the speech to text translator can translate the words spoken by the medical professional into a text based medical record which is subsequently stored in the electronic memory in the personal electronic device.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (I) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a speech to text transcription device according to an embodiment of the disclosure.

FIG. 2 is a front view of a housing of an embodiment of the disclosure.

FIG. 3 is a back view of a housing of an embodiment of the disclosure.

FIG. 4 is a top view of a housing of an embodiment of the disclosure.

FIG. 5 is a front view of a personal electronic device of an embodiment of the disclosure.

FIG. 6 is a perspective in-use view of an embodiment of the disclosure.

FIG. 7 is a schematic view of an embodiment of the disclosure.

### (J) DETAILED DESCRIPTION OF THE INVENTION

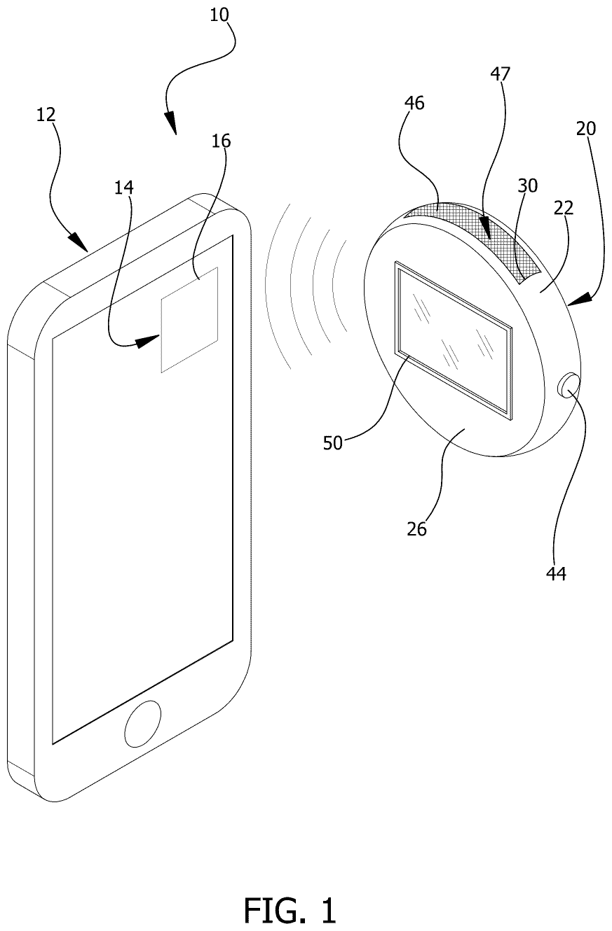

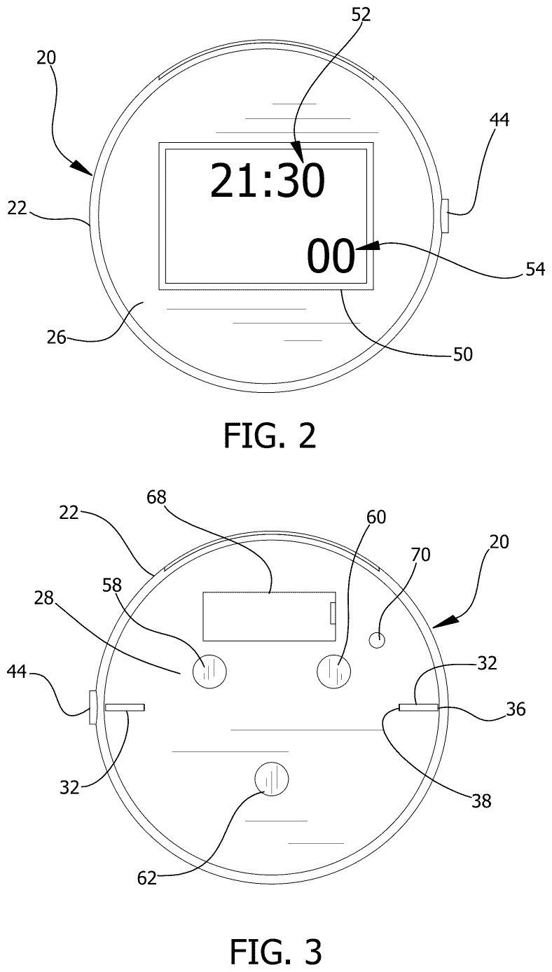

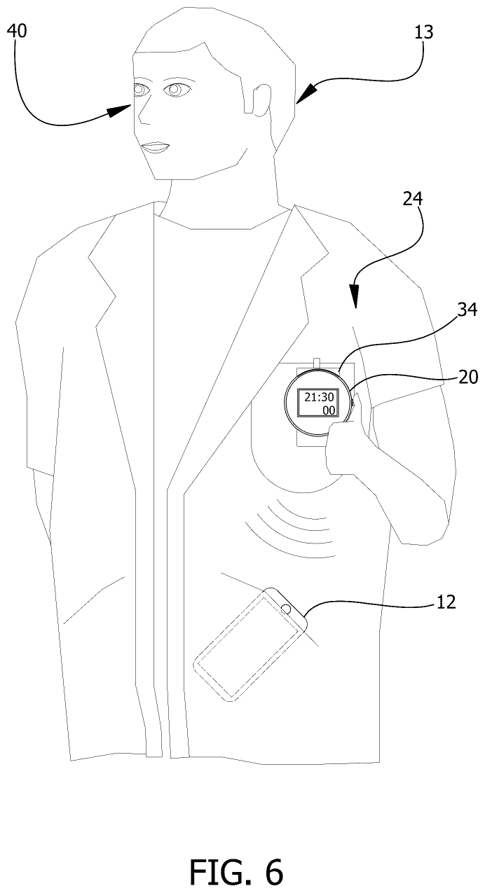

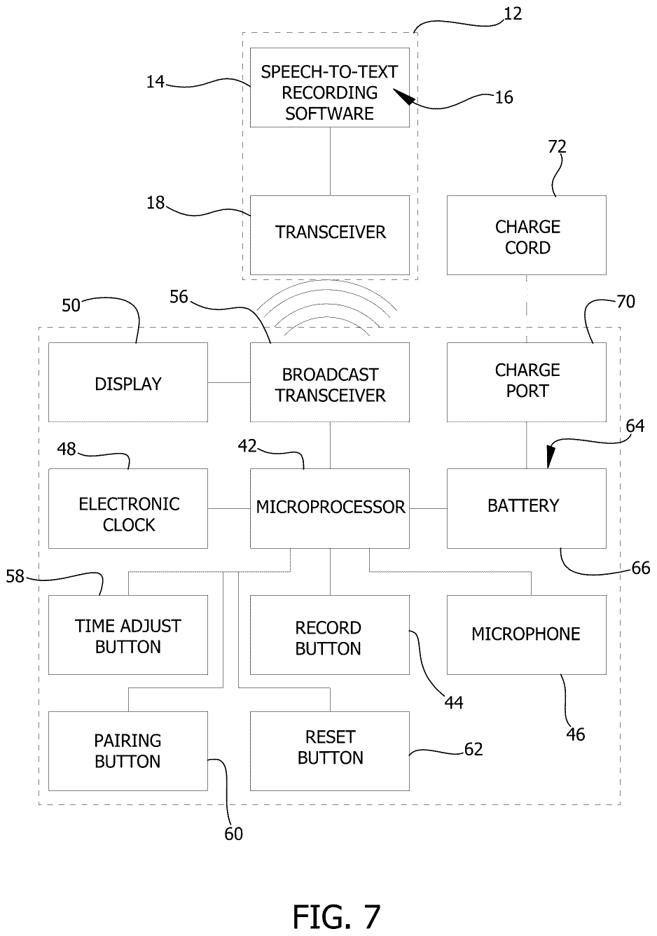

With reference now to the drawings, and in particular to FIGS. 1 through 7 thereof, a new transcription device embodying the principles and concepts of an embodiment of the disclosure and generally designated by the reference numeral 10 will be described.

As best illustrated in FIGS. 1 through 7, the speech to text transcription device 10 generally comprises a personal electronic device 12 which is carried by a medical professional 13. The personal electronic device 12 has an electronic memory 14 and the electronic memory 14 stores a database comprising a speech to text translator 16. The personal electronic device 12 includes a transceiver 18 that is integrated into the personal electronic device 12 and the transceiver 18 is in electrical communication with the electronic memory 14. The transceiver 18 may comprise a radio frequency transceiver or the like and the transceiver 18 may employ Bluetooth communication protocols. Furthermore, the personal electronic device 12 may comprise a smart phone or other similar type of electronic device that has wireless communication capabilities.

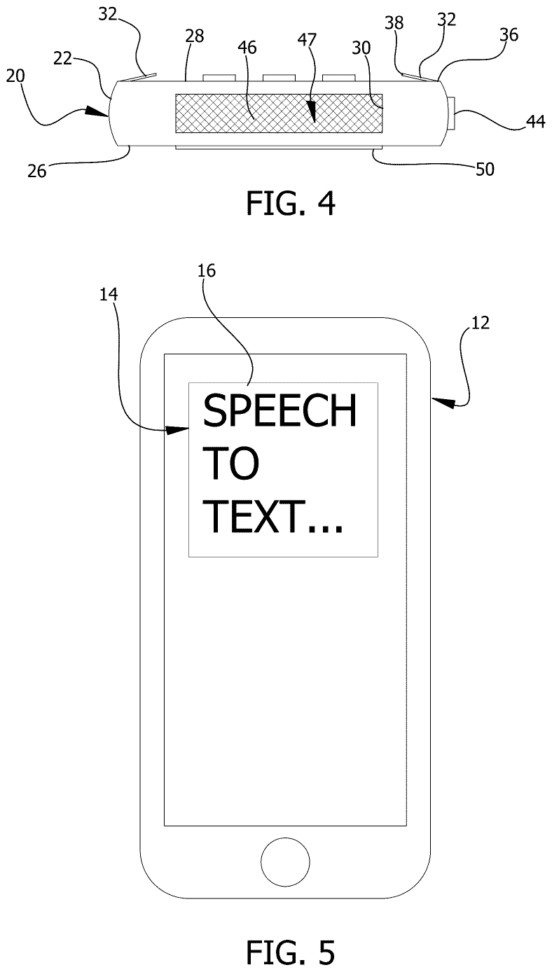

A housing 20 is provided which has a perimeter wall 22 that is continuously arcuate about a center of the housing 20 such that the housing 20 has a disk shape such that the housing 20 can be worn on clothing 24 of the medical professional 13 without catching on the clothing 24. The housing 20 has a front wall 26 and a back wall 28 and the perimeter wall 22 has a microphone opening 30 extending into an interior of the housing 20 to pass the audible sound of words spoken by the medical professional 13 into the housing 20. Furthermore, the microphone opening 30 is elongated along a circumference of the perimeter wall 22 such that the microphone opening 30 has a rectangular shape. As is most clearly shown in FIG. 4, the perimeter wall 22 may curve outwardly between the front wall 26 and the back wall 28.

A pair of clips 32 is each attached to the housing 20 such that each clip 32 of the pair of clips 32 can be attached to an identification badge 34 of the medical professional 13 for retaining the housing 20 on the identification badge 34 such that the housing 20 is not concealed in the clothing 24 of the medical professional 13. Each clip 32 of the pair of clips 32 has a coupled end 36 and a free end 38; the coupled end 36 of each clip 32 of the pair of clips 32 is coupled to the back wall 28 of the housing 20 at a point which is aligned with an intersection between the back wall 28 and the perimeter wall 22. Each clip 32 of the pair of clips 32 angles away from the back wall 28 such that the free end 38 of each clip 32 of the pair of clips 32 is spaced from the back wall 28. Furthermore, the identification badge 34 can be positioned between the pair of clips 32 and the back wall 28 of the housing 20. The pair of clips 32 is positioned on opposite sides of the back wall 28 such that the pair of clips 32 is directed toward each other. The microphone opening 30 in the perimeter wall 22 of the housing 20 is located between the pair of clips 32 such that the microphone opening 30 is directed upwardly toward a face 40 of the medical professional 13 when the pair of clips 32 is attached to the identification badge 34 to enhance the intensity and clarity of the audible sound of the words spoken by the medical professional 13 which pass through the microphone opening 30.

A processor 42 is located within the housing 20 and the processor 42 receives a record input. A record button 44 is movably integrated into the housing 20 such that the record button 44 can be depressed by the medical professional 13. The record button 44 is in electrical communication with the processor 42 and the processor 42 receives the record input when the record button 44 is depressed an initial time. Furthermore, the processor 42 is de-actuated when the record button 44 is depressed a subsequent time. The record button 44 is disposed on the perimeter wall 22 of the housing 20 such that the record button 44 is accessible to the medical professional 13 when the housing 20 is attached to the identification badge 34.

A microphone 46 is integrated into the housing 20 such that the microphone 46 can detect the audible sound of the words spoken by the medical professional 13. The microphone 46 is aligned with the microphone opening 30 in the perimeter wall 22 of the housing 20 such that the microphone 46 is exposed to the audible sound of the words spoken by the medical professional 13 that pass through the microphone opening 30. The microphone 46 is in electrical communication with the processor 42; the microphone 46 is actuated when the processor 42 receives the record input and the microphone 46 is de-actuated when the processor 42 is de-actuated. Furthermore, the microphone 46 may comprise an electronic microphone of any conventional design and, as is shown in FIGS. 1 and 4, the microphone 46 includes a mesh screen 47 to protect the microphone 46 from impact damage. An electronic clock 48 is integrated into the housing 20 and the electronic clock 48 is in electrical communication with the processor 42. The electronic clock 48 tracks a time of day and the electronic clock 48 has a timer function which is actuatable to count a duration of time that begins when the timer function is actuated. The timer function is actuated when the processor 42 receives the record input and the timer function is de-actuated when the processor 42 is de-actuated.

A display 50 is coupled to the front wall 26 of the housing 20 such that the display 50 is unobstructed when the housing 20 is worn by the medical professional 13. The display 50 is in electrical communication with the processor 42 and the display 50 displays clock indicia 52 comprising numbers to indicate the time of day tracked by the electronic clock 48. Additionally, the display 50 displays timer indicia 54 comprising numbers corresponding to the duration of time that is counted by the timer function of the electronic clock 48. The display 50 may comprise a light emitting diode display or other similar type of electronic display that can clearly display indicia in a darkened environment.

A broadcast transceiver 56 is integrated into the housing 20 and the broadcast transceiver 56 is in electrical communication with the processor 42. Furthermore, the broadcast transceiver 56 receives a signal from the microphone 46 such that the broadcast transceiver 56 can receive the words spoken by the medical professional 13. The broadcast transceiver 56 is in wireless communication with the transceiver 18 in the personal electronic device 12 and the broadcast transceiver 56 broadcasts the signal from the microphone 46 to the transceiver 18 in the personal electronic device 12. Furthermore, the transceiver 18 in the personal electronic device 12 receives the words spoken by the medical professional 13 such that the speech to text translator 16 can translate the words spoken by the medical professional 13 into a text based medical record which is subsequently stored in the electronic memory 14 in the personal electronic device 12.

The broadcast transceiver 56 is actuated to broadcast the time of day tracked by the electronic memory 14 to the transceiver 18 in the personal electronic device 12 when the processor 42 receives the record input. Furthermore, the speech to text translator 16 time stamps the text that is translated from the words spoken by the medical professional 13. The broadcast transceiver 56 is actuated to broadcast the signal from the microphone 46 when the processor 42 receives the record input. Conversely, the broadcast transceiver 56 ceases broadcasts the signal from the microphone 46 when the processor 42 is de-actuated. The broadcast transceiver 56 may comprise a radio frequency transceiver or the like and the broadcast transceiver 56 may employ Bluetooth communication protocols.

A time adjust button 58 is movably integrated into the back wall 28 of the housing 20. The time adjust button 58 is in electrical communication with the processor 42 such that the time adjust button 58 sets the time of day tracked by the electronic clock 48. A pairing button 60 is movably integrated into the back wall 28 of the housing 20 and the pairing button 60 is in electrical communication with the processor 42. Furthermore, the broadcast transceiver 56 is actuated to be paired with the transceiver 18 in the personal electronic device 12 when the pairing button 60 is depressed. A reset button 62 is movably integrated into the back wall 28 of the housing 20 and the reset button 62 is in electrical communication with the processor 42. The reset button 62 resets the timer function of the electronic clock 48 when the reset button 62 is depressed. A power supply 64 is positioned within the housing 20, the power supply 64 is in electrical communication with the processor 42 and the power supply 64 comprises at least one battery 66. A battery cover 68 may be removably attached to the back wall 28 of the housing 20 and the battery 66 may be positioned beneath the battery cover 68 to enable removal and replacement of the battery 66. Furthermore, the power supply 64 may include a charge port 70 that is recessed into the back wall 28 of the housing 20 which could be matable to a charge cord 72 for charging the battery 66.

In use, the pairing button 60 is depressed to pair the broadcast transceiver 56 with the transceiver 18 in the personal electronic device 12 and the pair clips 32 is each attached to the identification badge 34 of the medical professional 13, or any other object that is exposed on the clothing 24 of the medical professional 13, such that the microphone 46 is unobstructed. The record button 44 is depressed when the medical professional 13 needs to transcribe their spoken words into the text based medical record that is stored in the personal electronic device 12. Furthermore, the medical professional 13 can transcribe their spoken words into the text based medical record while the medical professional 13 is performing a medical procedure on the patient, for example, rather than requiring the medical professional 13 to manually create the text based medical record after the medical procedure has been completed. It is common that the medical professional 13 may struggle to accurately recall all of the details of the medical procedure after the medical procedure has been completed. The real time translation of the spoken words of the medical professional 13 into the text based medical record which is stored in the personal electronic device 12 ensures that the medical professional 13 can accurately create the text based medical record as the events of the medical procedure are occurring. Additionally, the personal electronic device 12 may have QR code scanning capabilities, for example, to enable the text based medical record that is stored in the electronic memory 14 of the personal electronic device 12 to be downloaded into the medical record of the patient subsequent to completion of the medical procedure. Furthermore, the electronic clock 48 enables the text based medical record to be time stamped while the medical record is being created in the speech to text translator 16 to ensure compliance with the Health Insurance Portability and Accountability Act.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A speech to text transcription device for converting spoken words of a medical professional into text which is stored in an electronic storage unit, said device comprising:
a personal electronic device having an electronic memory, said electronic memory storing a database comprising a speech to text translator;
a housing having a perimeter wall being continuously arcuate about a center of said housing such that said housing has a disk shape wherein said housing is configured to be worn on clothing of the medical professional without catching on the clothing;
a pair of clips, each clip of said pair of clips being attached to said housing wherein each clip of said pair of clips is configured to be attached to an identification badge of the medical professional for retaining said housing on the identification badge such that said housing is not concealed in the clothing of the medical professional;
a processor being located within said housing;
a record button being movably integrated into said housing wherein said record button is configured to be depressed by the medical professional, said record button being in electrical communication with said processor;
a microphone being integrated into said housing wherein said microphone is configured to detect the audible sound of the words spoken by the medical professional;
an electronic clock being integrated into said housing, said electronic clock being in electrical communication with said processor, said electronic clock tracking a time of day, said electronic clock having a timer function being actuatable to count a duration of time which begins when said timer function is actuated; and
a broadcast transceiver being integrated into said housing, said broadcast transceiver being in electrical communication with said processor such that said broadcast transceiver receives a signal from said microphone wherein said broadcast transceiver is configured to receive the words spoken by the medical professional, said broadcast transceiver being in wireless communication with said personal electronic device, said broadcast transceiver broadcasting said signal from said microphone to said personal electronic device when said record button is depressed wherein said personal electronic device is configured to receive the words spoken by the medical professional such that said speech to text translator can translate the words spoken by the medical professional into a text based medical record which is subsequently stored in said electronic memory in said personal electronic device.

2. The speech to text transcription device according to claim 1, wherein:
said housing has a front wall and a back wall;
said perimeter wall has a microphone opening extending into an interior of said housing wherein said microphone opening is configured to pass audible sounds into said housing; and
said microphone opening is elongated along a circumference of said perimeter wall such that said microphone opening has a rectangular shape.

3. The speech to text transcription device according to claim 2, wherein:
each clip of said pair of clips has a coupled end and a free end;
said coupled end of each clip of said pair of clips is coupled to said back wall of said housing at a point being aligned with an intersection between said back wall and said perimeter wall;
each clip of said pair of clips angles away from said back wall such that said free end of each clip of said pair of clips is spaced from said back wall wherein each clip of said pair of clips is configured to have the identification badge being positioned between said pair of clips and said back wall of said housing.

4. The speech to text transcription device according to claim 2, wherein:
said pair of clips is positioned on opposite sides of said back wall such that said pair of clips is directed toward each other; and
said microphone opening in said perimeter wall of said housing is located between said pair of clips wherein said microphone opening is configured to be directed upwardly toward a face of the medical professional when said pair of clips is attached to the identification badge to enhance the intensity and clarity of the audible sound of the words spoken by the medical professional which pass through said microphone opening.

5. The speech to text transcription device according to claim 1, wherein said record button is disposed on said perimeter wall of said housing wherein said record button is configured to be accessible to the medical professional when said housing is attached to the identification badge.

6. The speech to text transcription device according to claim 2, wherein:
said processor receives a record input when said record button is depressed an initial time;
said processor is de-actuated when said record button is depressed a subsequent time;
said microphone is aligned with said microphone opening in said perimeter wall of said housing wherein said microphone is configured to be exposed to the audible sound of the words spoken by the medical professional that pass through said microphone opening;
said microphone is in electrical communication with said processor;
said microphone is actuated when said processor receives said record input; and
said microphone is de-actuated when the processor is de-actuated.

7. The speech to text transcription device according to claim 1, wherein:
said processor receives a record input when said record button is depressed an initial time;
said processor is de-actuated when said record button is depressed a subsequent time;
said timer function is actuated when said processor receives said record input; and
said timer function is de-actuated when said processor is de-actuated.

8. The speech to text transcription device according to claim 2, wherein:
said speech to text transcription device includes a display being coupled to said front wall of said housing wherein said display is configured to be unobstructed when said housing is worn by the medical professional;
said display is in electrical communication with said processor;
said display displays clock indicia comprising numbers to indicate the time of day tracked by said electronic clock; and
said display displays timer indicia comprising numbers corresponding to said duration of time being counted by said timer function of said electronic clock.

9. The speech to text transcription device according to claim 1, wherein said broadcast transceiver broadcasts said time of day tracked by said electronic memory to said personal electronic device when said processor receives said record input wherein said speech to text translator is configured to time stamp the text based medical record that is translated from the words spoken by the medical professional.

10. The speech to text transcription device according to claim 1, wherein:
said processor receives a record input when said record button is depressed an initial time; and
said broadcast transceiver is actuated to broadcast said signal from said microphone when said processor receives said record input.

11. The speech to text transcription device according to claim 1, wherein:
said processor receives a record input when said record button is depressed an initial time;
said processor is de-actuated when said record button is depressed a subsequent time; and
said broadcast transceiver ceases broadcasting said signal from said microphone when said processor is de-actuated.

12. The speech to text transcription device according to claim 2, wherein:
said speech to text transcription device includes a time adjust button being movably integrated into said back wall of said housing; and
said time adjust button is in electrical communication with said processor such that said time adjust button sets the time of day tracked by said electronic clock.

13. The speech to text transcription device according to claim 2, wherein:
said speech to text transcription device includes a pairing button being movably integrated into said back wall of said housing;
said pairing button is in electrical communication with said processor; and
said broadcast transceiver is actuated to be paired with said personal electronic device when said pairing button is depressed.

14. The speech to text transcription device according to claim 2, wherein:
said speech to text transcription device includes a reset button being movably integrated into said back wall of said housing;
said reset button is in electrical communication with said processor; and
said reset button resets said timer function of said electronic clock when said reset button is depressed.

15. A speech to text transcription device for converting spoken words of a medical professional into text which is stored in an electronic storage unit, said device comprising:
a personal electronic device having an electronic memory, said electronic memory storing a database comprising a speech to text translator;
a housing having a perimeter wall being continuously arcuate about a center of said housing such that said housing has a disk shape wherein said housing is configured to be worn on clothing of the medical professional without catching on the clothing, said housing having a front wall and a back wall, said perimeter wall having a microphone opening extending into an interior of said housing wherein said microphone opening is configured to pass the audible sound of words spoken by the medical professional into said housing, said microphone opening being elongated along a circumference of said perimeter wall such that said microphone opening has a rectangular shape;
a pair of clips, each clip of said pair of clips being attached to said housing wherein each clip of said pair of clips is configured to be attached to an identification badge of the medical professional for retaining said housing on the identification badge such that said housing is not concealed in the clothing of the medical professional, each clip of said pair of clips having a coupled end and a free end, said coupled end of each clip of said pair of clips being coupled to said back wall of said housing at a point being aligned with an intersection between said back wall and said perimeter wall, each clip of said pair of clips angling away from said back wall such that said free end of each clip of said pair of clips is spaced from said back wall wherein each clip of said pair of clips is configured to have the identification badge being positioned between said pair of clips and said back wall of said housing, said pair of clips being positioned on opposite sides of said back wall such that said pair of clips is directed toward each other, said microphone opening in said perimeter wall of said housing being located between said pair of clips wherein said microphone opening is configured to be directed upwardly toward a face of the medical professional when said pair of clips is attached to the identification badge to enhance the intensity and clarity of the audible sound of the words spoken by the medical professional which pass through said microphone opening;
a processor being located within said housing, said processor receiving a record input;
a record button being movably integrated into said housing wherein said record button is configured to be depressed by the medical professional, said record button being in electrical communication with said processor, said processor receiving said record input when said record button is depressed an initial time, said record button being disposed on said perimeter wall of said housing wherein said record button is configured to be accessible to the medical professional when said housing is attached to the identification badge, said processor being de-actuated when said record button is depressed a subsequent time;
a microphone being integrated into said housing wherein said microphone is configured to detect the audible sound of the words spoken by the medical professional, said microphone being aligned with said microphone opening in said perimeter wall of said housing wherein said microphone is configured to be exposed to the audible sound of the words spoken by the medical professional that pass through said microphone opening, said microphone being in electrical communication with said processor, said microphone being actuated when said processor receives said record input, said microphone being de-actuated when the processor is de-actuated;
an electronic clock being integrated into said housing, said electronic clock being in electrical communication with said processor, said electronic clock tracking a time of day, said electronic clock having a timer function being actuatable to count a duration of time which begins when said timer function is actuated, said timer function being actuated when said processor receives said record input, said timer function being de-actuated when said processor is de-actuated;
a display being coupled to said front wall of said housing wherein said display is configured to be unobstructed when said housing is worn by the medical professional, said display being in electrical communication with said processor, said display displaying clock indicia comprising numbers to indicate the time of day tracked by said electronic clock, said display displaying timer indicia comprising numbers corresponding to said duration of time being counted by said timer function of said electronic clock;
a broadcast transceiver being integrated into said housing, said broadcast transceiver being in electrical communication said with processor such that said broadcast transceiver receives a signal from said microphone wherein said broadcast transceiver is configured to receive the words spoken by the medical professional, said broadcast transceiver being in wireless communication with said personal electronic device, said broadcast transceiver broadcasting said signal from said microphone to said personal electronic device wherein said personal electronic device is configured to receive the words spoken by the medical professional such that said speech to text translator can translate the words spoken by the medical professional into a text based medical record which is subsequently stored in said electronic memory in said personal electronic device, said broadcast transceiver being actuated to broadcast said time of day tracked by said electronic memory to said personal electronic device when said processor receives said record input wherein said speech to text translator is configured to time stamp the text based medical record that is translated from the words spoken by the medical professional, said broadcast transceiver being actuated to broadcast said signal from said microphone when said processor receives said record input, said broadcast transceiver ceasing broadcasting said signal from said microphone when said processor is de-actuated;
a time adjust button being movably integrated into said back wall of said housing, said time adjust button being in electrical communication with said processor such that said time adjust button sets the time of day tracked by said electronic clock;
a pairing button being movably integrated into said back wall of said housing, said pairing button being in electrical communication with said processor, said broadcast transceiver being actuated to be paired with said personal electronic device when said pairing button is depressed;
a reset button being movably integrated into said back wall of said housing, said reset button being in electrical communication with said processor, said reset button resetting said timer function of said electronic clock when said reset button is depressed; and
a power supply being positioned within said housing, said power supply being in electrical communication with said processor, said power supply comprising at least one battery.
